(19) United States
(12) Patent Application Publication
(10) Pub. No.: US 2023/0323479 A1
(43) Pub. Date: Oct. 12, 2023

(54) MOLECULAR MARKER OF CADMIUM ACCUMULATION IN RICE AND USE THEREOF IN IMPROVING CADMIUM ACCUMULATION IN RICE GRAINS

(71) Applicants: HUNAN HYBRID RICE RESEARCH CENTER, Changsha City (CN); HUNAN ACADEMY OF AGRICULTURAL SCIENCES, Changsha City (CN); YUEYANG AGRICULTURAL RESEARCH INSTITUTE, Yueyang City (CN)

(72) Inventors: Li LI, Changsha City (CN); Tiankang WANG, Changsha City (CN); Shufeng SONG, Changsha City (CN); Yixing LI, Changsha City (CN); Yinghong YU, Changsha City (CN); Lianyang BAI, Changsha City (CN); Yuefeng FU, Yueyang City (CN)

(21) Appl. No.: 18/024,593

(22) PCT Filed: Dec. 25, 2020
(86) PCT No.: PCT/CN2020/139294

§ 371 Date: Mar. 3, 2023

(30) Foreign Application Priority Data
Sep. 4, 2020 (CN) 202010919701.0

(51) Int. Cl. C12Q 1/6895

(52) U.S. Cl. CPC C12Q 1/6895; C12Q 2600/13; C12Q 2600/156

(57) ABSTRACT

A molecular marker of cadmium accumulation in rice improves cadmium accumulation in rice grains. The molecular marker of cadmium accumulation in rice includes a DNA fragment shown in the 8899129-9307609 region of rice chromosome 7, which is lcrf1 or lcrf2, lcrf1 is a DNA fragment shown in the 8899129-9307609 of rice chromosome 7, and the lcrf2 is the DNA fragment shown in SEQ ID No.1 in the sequence listing. The present invention finds that the molecular marker of cadmium accumulation in rice is related to the cadmium accumulation in rice grains, and the homozygous rice whose molecular marker of cadmium accumulation in rice is SEQ ID No. 1 in the sequence listing has the characteristics of low grain cadmium accumulation when planted in high cadmium polluted fields. Therefore, the DNA fragment can be introduced into other backgrounds by breeding methods such as hybridization and backcrossing.

### FIELD OF THE INVENTION

The present invention relates to the molecular marker of cadmium accumulation in rice and use thereof in improving cadmium accumulation in rice grains in the field of biotechnology.

### BACKGROUND OF THE INVENTION

Rice is one of the most important food crops in the world, and more than half of the world's population currently uses rice as a staple food. Cadmium is carcinogenic to the human body and has been listed as a Class I carcinogen by the International Agency for Research on Cancer. The main sources of cadmium in the soil are industrial waste gas, the deposition of automobile exhaust, the use of pesticides, chemical fertilizers and agricultural film, sewage irrigation, etc.; rice mainly absorbs cadmium in the soil through the roots, and the cadmium is absorbed by the roots and reaches vascular bundles through two transport pathways of symplast and apoplast, then transports to stems and leaves, and then migrates to grains, and finally accumulates in the human body through the food chain cycle, thus causing serious harm to human health.

At present, there are mainly the following four ways to reduce the cadmium content in rice grains. The first is to remove cadmium in soil or change the existing form of cadmium in soil by physical and chemical methods. The second is to inhibit the formation of soil exchangeable cadmium through cultivation measures such as water and fertilizer management. The third is to screen varieties with low grain cadmium accumulation from existing rice varieties. The fourth is to obtain new materials with low grain cadmium accumulation through gene editing technology. However, these four methods have great limitations in application. For example, the cost of remediation of cadmium-polluted soil is high, and it is easy to cause secondary pollution to the soil; different soil types or rice varieties require different cultivation measures, and the application effect is greatly affected by environmental conditions; the screening of varieties with low cadmium accumulation requires many years of multi-point verification, and many of the screened varieties will have high accumulation of cadmium in grains in heavily cadmium-polluted fields; although new materials with low cadmium accumulation can be obtained by gene editing technology in heavily cadmium-polluted fields, the country has not released the use of gene editing technology in production.

Cadmium accumulation in rice grains is a complex trait that is easily affected by the external environment and cultivation measures. Therefore, it is necessary to verify whether rice grains are low in cadmium accumulation through multiple points over many years, which brings great difficulties to the breeding of new rice varieties with cadmium accumulation in grains by hybridization and backcrossing. Therefore, it has become an urgent problem to be solved in production to explore an economical, rapid and efficient method for breeding new rice varieties with low grain cadmium accumulation.

### SUMMARY OF THE INVENTION

The technical problem to be solved by the present invention is how to economically, quickly and efficiently breed new rice varieties with low grain cadmium accumulation.

In order to solve the problems of the technologies described above, the present invention firstly provides any of the following applications:
1. Use of a substance for detecting a molecular marker of cadmium accumulation in rice in identifying or assisting in the identification of low cadmium accumulation traits in rice grains; the molecular marker of cadmium accumulation in rice is a DNA fragment in a 8899129-9307609 region of a rice chromosome 7(the reference genome is the CANU version of Shuhui 498 (updated on Nov. 23, 2018, website: http://www.mbkbase.org/R498/), and the molecular marker of cadmium accumulation in rice is lcrf1 or lcrf2 or lcrf3, and the lcrf1 is M1) or M2):
M1) a DNA fragment shown in the 8899129-9307609 of rice chromosome 7(the reference genome is the CANU version of Shuhui 498 (updated on Nov. 23, 2018, website: http://www.mbkbase.org/R498/), M2) a DNA fragment that undergoes substitution and/or deletion and/or addition of one or several nucleotides or DNA fragments to M1) and has 75% or more identity to M1);
the lcrf2 is N1) or N2):
N1) a DNA fragment shown in SEQ ID No.1 in the sequence listing, N2) a DNA fragment that undergoes substitution and/or deletion and/or addition of one or several nucleotides or DNA fragments to N1) and has 75% or more identity to N1);
the lcrf3 is O1) or O2):
O1) a DNA fragment shown in SEQ ID No.2 in the sequence listing, O2) a DNA fragment that undergoes substitution and/or deletion and/or addition of one or several nucleotides or DNA fragments to O1) and has 75% or more identity to O1);
2. The use of a substance for detecting a molecular marker of cadmium accumulation in rice in the preparation of a product for identifying or assisting in the identification of low cadmium accumulation traits in rice grains.
3. The use of a substance for detecting a molecular marker of cadmium accumulation in rice in rice breeding or preparing a rice breeding product.

Those skilled in the art can easily use known methods, such as directed evolution and point mutation methods, to mutate the nucleotide sequences of MD, Ni) and O1) of the present invention. Those artificially modified nucleotides having 75% or higher identity with the nucleotide sequences of MD, Ni) and O1) of the present invention are all derived from the nucleotide sequences of the present invention and are equivalent to sequences of the present invention.

The term “identity” as used herein refers to sequence similarity to a native nucleic acid sequence. “Identity” includes 75% or higher, 80% or higher, or 85% or higher, or 90% or higher, or 95% or higher, or 97% or higher, or 98% or higher, or 99% or higher identity with the nucleotide sequences of M1), N1) and O1) of the present invention. Identity can be assessed visually or with computer software. Using computer software, identity between two or more sequences can be expressed as a percentage (%), which can be used to evaluate the identity between related sequences.

In the above uses, the substance for detecting a molecular marker of cadmium accumulation in rice is a substance that can detect the molecular marker of cadmium accumulation in rice by conventional experimental methods and can specifically recognize the molecular marker of cadmium accumulation in rice, and the substance can be PCR primers and/or probes that can distinguish molecular marker of cadmium accumulation in rice as the lcrf1 and the lcrf2, as long as they can specifically identify the molecular marker of cadmium accumulation in rice of the present invention, all fall within the scope of protection of the present invention.

The PCR primer can specifically be part or all of the following (a1), (a2), (a3) and (a4):
(a1) is (b1) or (b2) or (b3) as follows:
(b1) a single-stranded DNA molecule shown in SEQ ID No.3 of the sequence listing;
(b2) a single-stranded DNA molecule that undergoes substitution and/or deletion and/or addition of one or several nucleotides to SEQ ID No. 3 and has 75% or more identity to (b1);
(b3) a DNA molecule that hybridizes under stringent conditions to the nucleotide sequence defined in (b1) or (b2);
(a2) is (c1) or (c2) or (c3) as follows:
(c1) a single-stranded DNA molecule shown in SEQ ID No.4 of the sequence listing;
(c2) a single-stranded DNA molecule that undergoes substitution and/or deletion and/or addition of one or several nucleotides to SEQ ID No. 4 and has 75% or more identity to (c1);
(c3) a DNA molecule that hybridizes under stringent conditions to the nucleotide sequence defined in (c1) or (c2);
(a3) is (d1) or (d2) or (d3) as follows:
(d1) a single-stranded DNA molecule shown in SEQ ID No.5 of the sequence listing;
(d2) a single-stranded DNA molecule that undergoes substitution and/or deletion and/or addition of one or several nucleotides to SEQ ID SEQ ID No.5 and has 75% or more identity to (d1);
(d3) a DNA molecule that hybridizes under stringent conditions to the nucleotide sequence defined in (d1) or (d2);
(a4) is (e1) or (e2) or (e3) as follows:
(e1) a single-stranded DNA molecule shown in SEQ ID No. 6 of the sequence listing;
(e2) a single-stranded DNA molecule that undergoes substitution and/or deletion and/or addition of one or several nucleotides to SEQ ID No. 6 and has 75% or more identity to (e1);
(e3) a DNA molecule that hybridizes under stringent conditions to the nucleotide sequence defined in (e1) or (e2).

Those skilled in the art can easily use known methods, such as directed evolution and point mutation methods, to mutate the nucleotide sequences of (b1), (e1), (d1) and (e1) of the present invention. Those artificially modified nucleotides having 75% or higher identity with the nucleotide sequences of (b1), (e1), (d1) and (e1) of the present invention are all derived from the nucleotide sequences of the present invention and are equivalent to sequences of the present invention.

The term “identity” as used herein refers to sequence similarity to a native nucleic acid sequence. “Identity” includes 75% or higher, 80% or higher, or 85% or higher, or 90% or higher, or 95% or higher, or 97% or higher, or 98% or higher, or 99% or higher identity with the nucleotide sequences of (b1), (c1), (d1) and (e1) of the present invention. Identity can be assessed visually or with computer software. Using computer software, identity between two or more sequences can be expressed as a percentage (%), which can be used to evaluate the identity between related sequences.

The stringent conditions can be as follows: hybridization at 50° C. in a mixture of 7% sodium dodecyl sulfate (SDS), 0.5M NaPO₄ and 1 mM EDTA, rinsing at 50° C. in 2×SSC, 0.1% SDS; it can also be: hybridization at 50° C. in a mixture of 7% SDS, 0.5M NaPO₄ and 1 mM EDTA, rinsing at 50° C. in 1×SSC, 0.1% SDS; it can also be: hybridization at 50° C. in a mixture of 7% SDS, 0.5M NaPO₄ and 1 mM EDTA, rinsing at 50° C. in 0.5×SSC and 0.1% SDS; it can also be: hybridization at 50° C. in a mixture of 7% SDS, 0.5 M NaPO₄ and 1 mM EDTA, rinsing at 50° C. in 0.1×SSC, 0.1% SDS; it can also be: hybridization at 50° C. in a mixture of 7% SDS, 0.5 M NaPO₄ and 1 mM EDTA, rinsing at 65° C. in 0.1×SSC, 0.1% SDS; it can also be: hybridization at 65° C. in 6×SSC, 0.5% SDS solution, and then washing with 2×SSC, 0.1% SDS and 1×SSC, 0.1% SDS once each; it can also be: hybridization at 68° C. in 2×SSC, 0.1% SDS solution and washing the membrane twice, 5 min each time, and hybridization at 68° C. in 0.5><SSC, 0.1% SDS solution and washing the membrane twice, 15 min each time; it can also be: hybridization at 65° C. in 0.1×SSPE (or 0.1×SSC) and 0.1% SDS solution and washing the membrane.

Using rice genomic DNA as a template, the rice to be tested which can be amplified to a DNA fragment of length 483 bp using the two single-stranded DNAs shown in SEQ ID Nos. 3 and 4 and which can be amplified to a DNA fragment of length 494 bp using the two single-stranded DNAs shown in SEQ ID Nos. 5 and 6 is heterozygous rice containing the lcrf1 and the lcrf2; using rice genomic DNA as a template, the rice to be tested which can be amplified to a DNA fragment of length 483 bp using the two single-stranded DNAs shown in SEQ ID Nos. 3 and 4 and which cannot be amplified to a DNA fragment of length 494 bp using the two single-stranded DNAs shown in SEQ ID Nos. 5 and 6 is homozygous rice containing the lcrf1; using rice genomic DNA as a template, the rice to be tested which cannot be amplified to a DNA fragment of length 483 bp using the two single-stranded DNAs shown in SEQ ID Nos. 3 and 4 and which can be amplified to a DNA fragment of length 494 bp using the two single-stranded DNAs shown in SEQ ID Nos. 5 and 6 is homozygous rice containing the lcrf2.

The present invention further provides a method for identifying or assisting in the identification of low cadmium accumulation traits in rice grains, and the method includes: detecting the DNA fragment in the 8899129-9307609 region of chromosome 7 of the rice to be tested, and determining the low grain cadmium accumulation traits of the rice to be tested according to the DNA fragment in the 8899129-9307609 region of chromosome 7 of the rice to be tested.

In the above method, the DNA fragment in the 8899129-9307609 region of rice chromosome 7 is the lcrf1 or the lcrf2; the cadmium content in grains of the homozygous rice containing the lcrf2 is lower or candidate lower than that of heterozyous rice containing the lcrf1 and the lcrf2, and the cadmium content in grains of homozygous rice containing the lcrf2 is lower or candidate lower than that of homozygous rice containing lcrf1.

The method for identifying or assisting in the identification of low cadmium accumulation traits in rice grains or the use of lcrf2 in rice breeding also falls within the protection scope of the present invention.

The present invention further provides a rice breeding method, and the method includes: detecting the DNA fragment in the 8899129-9307609 region of chromosome 7 in the rice genome, and selecting the DNA fragment in the 8899129-9307609 region of chromosome 7 as the homozygous or heterozygous rice for lcrf2 as a parent for breeding.

In the above method, the rice breeding is to cultivate rice with low grain cadmium accumulation.

During breeding, the lcrf2 can be introduced into other background rices by breeding methods such as hybridization or backcrossing to select rice with low grain cadmium accumulation.

The parent may be a male sterile line of rice. In one embodiment of the present invention, the parent is Luohong 3A or Luohong 4A.

The following X1 or X2 also fall into the protection scope of the present invention:
X1. the substance for detecting a molecular marker of cadmium accumulation in rice;
X2, the lcrf2.

In the present invention, the substance for detecting a molecular marker of cadmium accumulation in rice can be a reagent or a kit or a system. The system can include a combination product of reagents or kits, instruments and analysis software, such as a product comprising PCR primers, and reagents used for PCR amplification.

### BRIEF DESCRIPITION OF THE FIGURE

FIG. 1 shows partial results of mf-F and mf-R PCR detection. M is DNA molecular weight standard (band size from top to bottom: 2000 bp, 1500 bp, 1000 bp, 750 bp, 500 bp, 250 bp, 100 bp), 1-24 are different individual plants of F2 population.

FIG. 2 shows partial results of lcrf-F and lcrf-R PCR detection. M is DNA molecular weight standard (band size from top to bottom: 2000 bp, 1500 bp, 1000 bp, 750 bp, 500 bp, 250 bp, 100 bp), 1-16 are different individual plants of F2 population.

### DETAILED DESCRIPTION

The present invention will be further described in detail below in conjunction with specific embodiments, and the given examples are only for clarifying the present invention, not for limiting the scope of the present invention. The examples provided below can be used as a guideline for those skilled in the art to make further improvements, and are not intended to limit the present invention in any way.

The experimental methods in the following examples, unless otherwise specified, are conventional methods and are carried out according to the techniques or conditions described in the literature in the art or according to the product instructions. The materials, reagents, apparatus and the like used in the following embodiments are commercially available if not otherwise specified. Quantitative experiments in the following examples are all set up to repeat the experiments three times, and the results are averaged. In the following examples, unless otherwise specified, the first position of each nucleotide sequence in the sequence listing is the 5′ terminal nucleotide of the corresponding DNA/RNA, and the last position is the 3′ terminal nucleotide of the corresponding DNA/RNA.

Luohong 3A in the following examples (Zhu Renshan, Liu Wenjun, Li Shaoqing, Zhu Yingguo, Breeding and utilization of red lotus type hybrid rice sterile line Luohong 3A and its combination Luoyou 8, Journal of Wuhan University (Science Edition), Vol. 59, No. 1, February 2013), the public can obtain the biological material from the applicant, and the biological material is only used for repeating the relevant experiments of the present invention, and cannot be used for other purposes.

Luohong 4A in the following examples (Zhu Renshan, Huang Wenchao, Hu Jun, Liu Wenjun, Zhu Yingguo, Breeding of Luohong 4A, a new sterile line of red lotus type hybrid rice, Journal of Wuhan University (Science Edition), Vol. 59, No. 1, February 2013), the public can obtain the biological material from the applicant, and the biological material is only used for repeating the relevant experiments of the present invention, and cannot be used for other purposes.

Minghui 63 in the following examples (Wu Fangxi, Cai Qiuhua, Zhu Yongsheng, et al. Utilization and innovation of indica hybrid rice restorer line Minghui 63 [J]. Fujian Agricultural Journal, 2011(06):1101-1112.), the public can obtain the biological material from the applicant, and the biological material is only used for repeating relevant experiments of the present invention, and cannot be used for other purposes.

Longtefu B in the following examples (Hu Dehui, Liu Kaiyu, Zhou Ping, et al. Molecular marker-assisted selection to improve rice quality of Tian B and Longtefu B[J]. Molecular Plant Breeding Printed Edition, 2013, 11(005) :486-493.), the public can obtain the biological material from the applicant, and the biological material is only used for repeating the relevant experiments of the present invention, and cannot be used for other purposes.

Huanghuazhan in the following examples (Pan Fuhe, Wu Duoxin. High-yielding cultivation techniques for new high-quality conventional rice variety Huanghuazhan [J]. Modern Agricultural Science and Technology, 2009, 000(001):193-193.), the public can obtain the biological material from the applicant, and the biological material is only used for repeating the relevant experiments of the present invention, and cannot be used for other purposes.

The detection method of cadmium content in grains: After wet digestion with nitric acid-perchloric acid (4:1, V/V), the metal content of rice grain was measured by AA240FS graphite furnace atomic absorption spectrophotometer (VARIAN, USA). The quality control was carried out with the Chinese national standard material GB W080684 as the internal standard, and blank experiments were carried out throughout the process. All utensils were soaked in 5% nitric acid solution overnight and rinsed with deionized water.

Gold Medal Mix: Beijing Qingke Biotechnology Co., Ltd.; Item No.: TSE101.

### EXAMPLE 1, DISCOVERY OF A MOLECULAR MARKER OF CADMIUM ACCUMULATION IN RICE

### 1. Screening Materials with Low Grain Cadmium Accumulation in Existing Rice Resources

At present, several rice hybrid combinations with emergency low cadmium accumulation have been reported, but these emergency combinations were still highly accumulated in grains planted in medium and high cadmium polluted soil (higher than the upper limit of the national standard of 0.2 mg/kg). In order to seek rice materials with low accumulation of grain planted in medium and high cadmium contaminated soil, the inventors sown 275 rice resources (including 162 restorer lines, 35 sterile lines and 78 foreign materials) collected from China and abroad to moderate cadmium contaminated fields (total cadmium content in the soil was 0.8 mg/kg) to screen low grain cadmium accumulation varieties. For the sterile lines, other fertile materials were used as male parents to pollinate them to make them seed. The cadmium content in grains of each material was measured after harvest. It was found that compared with other parents, the cadmium content in grains of male sterile lines Luohong 3A and Luohong 4A were extremely low, which was 0.01 mg/kg and 0.03 mg/kg, respectively, while the cadmium content in grains of other rice materials was all higher than 0.20 mg/kg.

Then Luohong 3A, Luohong 4A and other materials were sown in heavily cadmium-polluted fields (total cadmium content in the soil was 5.0 mg/kg), and Luohong 3A and Luohong 4A were still pollinated with other fertile materials as male parents to make them seed. The cadmium content in grains of each material was measured after harvest. It was found that the cadmium content in grains of Luohong 3A and Luohong 4A was also extremely low, which was 0.03 mg/kg and 0.06 mg/kg, respectively, while the cadmium content in grains of other rice varieties was all higher than 0.90 mg/kg.

### 2. Whole Genome Sequencing of Luohong 4A

In order to obtain the reasons for the low grain cadmium accumulation of Luohong 4A and Luohong 3A in heavily cadmium-polluted fields from the genetic level, the inventors carried out third-generation high-depth sequencing, genome de novo assembly and comparative genomics analysis on Luohong 4A. It was found that in the 8899129-9307609 region of chromosome 7 of Luohong 4A (the reference genome was the CANU version of Shuhui 498, the update date was Nov. 23, 2018, website: http://www.mbkbase.org/R498/), namely a total of 408481 bp fragments (the fragment was named lcrf1) were replaced by a 2980 bp fragment (the fragment was named lcrf 2, whose sequence was SEQ ID No.1 in the sequence listing). The 8899129-9307609 region of chromosome 7 of Luohong 3A and its upstream and downstream were amplified by Sanger sequencing, and it was found that lcrf1 was also replaced by lcrf2.

### 3. Correlation Analysis Between lcrf2 Fragments and Cadmium Accumulation Characteristics in Rice Grains

Luohong 3A (as the female parent) was hybridized with Minghui 63, and 52 F1 plants were obtained by sowing after harvesting seeds. F1 plants were self-crossed and mixed to obtain F2 seeds. Some F2 seeds were sown to moderate cadmium contaminated fields (total cadmium content in the soil was 0.8 mg/kg) to obtain F2 populations. Genomic DNA was extracted from 1,000 individual plants after transplanting, and PCR amplification was carried out using the obtained genomic DNA as a template to detect the genotype of each individual plant, that is, the DNA fragments in the 8899129-9307609 region of chromosome 7.

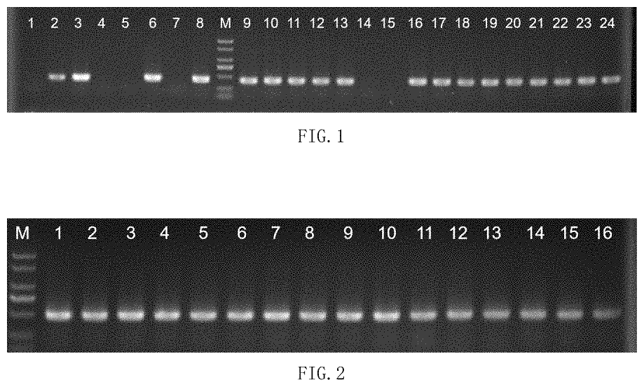

The detection method was as follows: the forward and reverse primers in the 8899129-9307609 region of chromosome 7 were designed (mf-F: 5′-ACTTGACAATCGATCCAACTAGC-3′ (SEQ ID No.3 in the sequence listing); mf-R: 5′-CGAAGCTTTGCTGATCGGG-3′ (SEQ ID No.4 in the sequence listing)), the full length of the amplified sequence was 483 bp. The PCR reaction system was 10 μl: Gold Mix 8.5 μl, mf-F 0.5 μl, mf-R 0.5 μl, template DNA 0.5 μl. The PCR amplification program was: pre-denaturation at 98° C. for 2 min; denaturation at 98° C. for 10 s, annealing at 54° C. for 10 s, extension at 72° C. for 10 s, 30 cycles, and extension at 72° C. for 5 min. Then electrophoresis was then carried out, and the electrophoresis results showed that some samples could amplify 483 bp bands, while some samples could not amplify 483 bp bands. Wherein, the samples that could amplify a 483 bp band were homozygous individual plant for lcrf1 or heterozygous individual plant for lcrf1/lcrf2 (see FIG. 1 for some detection results).

For samples that cannot amplify the 483 bp band, the next step of detection and verification was carried out, that is, forward and reverse primers (lcrf-F: 5′-CGCCGAATTGTAGGAGTTG -3′(SEQ ID No.5 in the sequence listing); lcrf-R: 5′-GGATGGTTTAGGTGGATGG -3′ (SEQ ID No.6 in the sequence listing)) were designed on the lcrf2 fragment and its flanking (named lcrf3, SEQ ID No.2 in the sequence listing) respectively. The full length of the amplified sequence was 494 bp. The PCR reaction system was 10 μl: Gold Mix 8.5 μl, lcrf-F 0.5 μl, lcrf-R 0.5 μl, template DNA 0.5 μl. The PCR amplification program was as follows: pre-denaturation at 98° C. for 2 min; denaturation at 98° C. for 10 s, annealing at 55° C. for 10 s, extension at 72° C. for 10 s, 30 cycles, and extension at 72° C. for 5 min. Then electrophoresis was then carried out, and the sample that could amplify a 494 bp band was a homozygous individual plant containing the lcrf2 fragment (see FIG. 2 for some detection results).

The samples that could amplify 483 bp bands were amplified by PCR using lcrf-F and lcrf-R. The samples that could amplify 494 bp bands were heterozygous individual plants containing lcrf1 and lcrf2 fragments, and the samples that could not amplify 494 bp bands were homozygous individual plants containing lcrf1 fragments.

For the detected homozygous individual plants containing lcrf2 fragments, heterozygous individual plants containing lcrf1 and lcrf2 fragments, and homozygous individual plants containing lcrf1 fragments, 10 were randomly selected for the 8899129-9307609 region of chromosome 7 (the reference genome was the CANU version of Shuhui 498, the update date was Nov. 23,2018, website: http://www.mbkbase.org/R498/) for sequencing. The results showed that the region of both chromosomes of the homozygous individual plants containing the lcrf2 fragment was the lcrf2 fragment, one of the region of both chromosomes of the heterozygous individual plants containing the lcrf1 and lcrf2 fragments was the lcrf2 fragment and one was the lcrf1 fragment, and the region of the two chromosomes of the homozygous individual plants containing the lcrf1 fragment was the lcrf1 fragment.

After the F2 population matured, 1000 grains of individual plants were harvested, and then the cadmium content of the grains of individual plants was detected. Combined with the genotype detection data, the final results showed that among the 1000 individual plants, there were 253 homozygous individual plants containing lcrf2, and the results of the cadmium content in grains of individual plant ranged from 0.01 to 0.04 mg /kg, with an average of 0.02 mg/kg; for 498 heterozygous individual plants containing lcrf1 and lcrf2, the results of the cadmium content in grains of individual plants ranged from 0.21 to 0.57 mg/kg, with an average of 0.39 mg/kg, which was significantly higher than that of homozygous individual plants containing lcrf2; for 249 homozygous individual plants containing lcrf1, the results of the cadmium content in grains of individual plant ranged from 0.23 to 0.63 mg/kg, with an average of 0.41 mg/kg, which was significantly higher than that of homozygous individual plants containing lcrf2. There was no significant difference in grains of cadmium content between heterozygous individual plants containing lcrf1 and lcrf2 and homozygous individual plants containing lcrf1.

Then another part of F2 seeds were sown in highly cadmium-polluted fields (total cadmium content in the soil was 3.5 mg/kg) to obtain F2 population 2. After transplanting, 1000 individual plants were still taken to extract genomic DNA, and the genotype of each individual plant was detected according to the above method. Then for the detected homozygous individual plants containing lcrf2 fragments, heterozygous individual plants containing lcrf1 and lcrf2 fragments, and homozygous individual plants containing lcrf1 fragments, 10 were randomly selected for the 8899129-9307609 region of chromosome 7 (the reference genome was the CANU version of Shuhui 498, the update date was Nov. 23,2018, website: http://www.mbkbase.org/R498/) for sequencing. The results showed that the region of both chromosomes of the homozygous individual plants containing the lcrf2 fragment was the lcrf2 fragment, one of the region of both chromosomes of the heterozygous individual plants containing the lcrf1 and lcrf2 fragments was the lcrf2 fragment and one was the lcrf1 fragment, and the region of the two chromosomes of the homozygous individual plants containing the lcrf1 fragment was the lcrf1 fragment. After the F2 population 2 matured, 1000 grains of individual plants were harvested, and then the cadmium content of the grains of individual plants was detected. The final results showed that among the 1000 individual plants, there were 255 homozygous individual plants containing lcrf2, and the results of the cadmium content in grains of individual plant ranged from 0.01 to 0.07 mg /kg, with an average of 0.05 mg/kg; for 502 heterozygous individual plants containing lcrf1 and lcrf2, the results of the cadmium content in grains of individual plants ranged from 0.89 to 6.57 mg/kg, with an average of 3.23 mg/kg, which was significantly higher than that of homozygous individual plants containing lcrf2; for 243 homozygous individual plants containing lcrf1, the results of the cadmium content in grains of individual plant ranged from 0.81 to 6.62 mg/kg, with an average of 3.27 mg/kg, which was significantly higher than that of homozygous individual plants containing lcrf2. There was no significant difference in grains of cadmium content between heterozygous individual plants containing lcrf1 and lcrf2 and homozygous individual plants containing lcrf1.

The above studies showed that the replacement of lcrf1 by lcrf2 in the 8899129-9307609 region of chromosome 7 was the reason for the low grain cadmium accumulation of rice in heavily cadmium-polluted fields. These two fragments could be used as molecular marker of cadmium accumulation in rice for rice breeding.

### EXAMPLE 2, THE USE OF MOLECULAR MARKER OF CADMIUM ACCUMULATION IN RICE IN RICE BREEDING

### 1 Preparation of Rice Low Cadmium Material DGHJ

Luohong 4A (as a female parent) was hybridized with Huanghuazhan. The F 1 population was obtained by sowing after harvesting, and the F1 population was backcrossed with Huanghuazhan. The BC1F1 population was obtained by sowing after harvesting, and the genomic DNA was extracted from the individual plant of the BC1F1 population. The primers lcrf-F/lcrf-R of the Example 1 were used for PCR amplification. The individual plant corresponding to the sample that could amplify the 494 bp band was backcrossed with Huanghuazhan; the BC2F1 population was obtained by sowing after harvesting, and the genomic DNA was extracted from the individual plant of the BC2F1 population. The primers lcrf-F/lcrf-R of the Example 1 were used for PCR amplification. The individual plant corresponding to the sample that could amplify the 494 bp band was backcrossed with Huanghuazhan. The BC3F1 population was obtained by resowing after harvesting, and the genomic DNA was extracted from the individual plant of the BC3F1 population. The primers lcrf-F/lcrf-R of the Example 1 were used for PCR amplification. The individual plant corresponding to the sample that could amplify the 494 bp band was backcrossed with Huanghuazhan. The BC4F1 population was obtained by resowing after harvesting, and the genomic DNA was extracted from the individual plant of the BC4F1 population. The primers lcrf-F/lcrf-R of Example 1 were used for PCR amplification. The individual plants corresponding to the samples that could amplify the 494 bp band were anther cultured. After the seedlings were obtained, the genomic DNA was extracted. The obtained genomic DNA was first amplified by PCR with the primers mf-F/mf-R of Example 1, and the samples that could not amplify the 483 bp band were then used for PCR amplification with the primers lcrf-F/lcrf-R of Example 1. The individual plant corresponding to the sample that could amplify the 494 bp band, which was a rice diploid low cadmium new material DGHJ with homozygous lcrf2 fragment and similar to Huanghuazhan.

8899129-9307609 region of chromosome 7 of the low cadmium new material DGHJ (the reference genome was the CANU version of Shuhui 498, the update date was Nov. 23, 2018, website: http://www.mbkbase.org/R498/) for sequencing. The results showed that the region of both chromosomes was lcrf2 fragment.

Then the low cadmium material DGHJ was sown in heavily cadmium-polluted fields (total cadmium content in the soil was 5.0 mg/kg), and Huanghuazhan was sown at the same time as a control. The cadmium content of the grains was harvested after maturity, and the cadmium content of DGHJ grains was found to be 0.04 mg/kg, while the cadmium content in grains of Huang Huazhan was 2.15 mg/kg.

### 2 Preparation of Rice Low-Cadmium New Material DGZJ

Luohong 3A (as female parent) was hybridized with Longtefu B. The F1 population was obtained by sowing after harvesting, and the F2 population was obtained by sowing after harvesting, and the genomic DNA was extracted from the individual plant of the F2 population. The primers lcrf-F/lcrf-R of the Example 1 were used for PCR amplification. The individual plant corresponding to the sample that could amplify the 494 bp band was harvested; and then the F3 population was obtained by sowing, and the genomic DNA was extracted from the individual plant of the F3 population. The primers lcrf-F/lcrf-R of the Example 1 were used for PCR amplification. The individual plant corresponding to the sample that could amplify the 494 bp band was harvested, and then the F4 population was obtained by resowing, and the genomic DNA was extracted from the individual plant of the F4 population. The primers lcrf-F/lcrf-R of the Example 1 were used for PCR amplification. The individual plant corresponding to the sample that could amplify the 494 bp band was harvested, and then the F4 population was obtained by resowing, and the genomic DNA was extracted from the individual plant of the F4 population. The primers lcrf-F/lcrf-R of the Example 1 were first used for PCR amplification. The sample that could not amplify the 483 bp band was then amplified by PCR with the primer lcrf-F/lcrf-R of Example 1. The individual plant corresponding to the sample that could amplify the 494 bp band was a homozygous individual plant containing the lcrf2 fragment, and the homozygous individual plant containing the lcrf2 fragment was then self-crossed to obtain a stable low-cadmium new material DGZJ with excellent comprehensive traits.

8899129-9307609 region of chromosome 7 of the low cadmium new material DGZJ (the reference genome was the CANU version of Shuhui 498, the update date was Nov. 23, 2018, website: http://www.mbkbase.org/R498/) for sequencing. The results showed that the region of both chromosomes was lcrf2 fragment.

Then the low cadmium material DGZJ was sown in heavily cadmium-polluted fields (total cadmium content in the soil was 5.0 mg/kg), and Longtefu B was sown at the same time as a control. The cadmium content of the grains was harvested after maturity, and the cadmium content of DGZJ grains was found to be 0.05 mg/kg, while the cadmium content in grains of Longtefu B was 2.73 mg/kg.

The sequences were as follows:

| SEQ ID No. 1: | |
|---|---|
| agctaagcct taactaaacc gcagcgggat caccactcca caggcatcct cgacggcacg | 60 |
| gacgaagcct actcctcaga gaaaccttca tctgacctga actcgaactc tggggttggg | 120 |
| aaaagtagag caagactgag tactacccac tgtactcagc aagtcatacc ggaatagagg | 180 |
| tatgatgcag ggaatcatca aaaaaaaaac taaagtggtt catttgcata aagcgagcat | 240 |
| ttataaacag aagttgaaag gtttaaacag ttgtaataat taatgattat taatcatccg | 300 |
| ctgtccaacg ctaacctacg ttgcgacagg cccaaccatc cacctaaacc atccaaatta | 360 |
| aaagattaca ctaaggtgag actaatcacg gtgaatctgg ttgaccgccc ataaccgcgg | 420 |
| gcacggctat tcgaatagtt ttactctgat cagaggtgta caactgtacc cacaagacac | 480 |
| agccccacga cacgtttccg tgcgccgaca tgtcaccacg acatatcgga aagaggccgt | 540 |
| gacaggacca ttcgcataac cccctctaac caagcacacc acacctcagg tttcaccccc | 600 |
| actcctcgca aggtagcggg cagtcccctc tcgtgcctag gtgaatccga aagccgcata | 660 |
| ggccgtcgca gggcccatcc aaactccatc acgcccaccc ttgcctggat gcgtctgcta | 720 |
| ggggaaagct acactacaag cccagccgtt gcccatgctg gcttgtggta agtacgttaa | 780 |
| gttcttccag ggcatcccgc gaaccggtcc ttaactgcca tgggtgcgac cagcaaaacc | 840 |
| atgcacccac agcccaccat gtgattcatt ttatttaacc aacaccagag cggtggtact | 900 |
| aatccaacat taccagaaga accaacagtc taaacattaa tatgattttc cccattgtga | 960 |
| actagttgaa ctaagcatgg ctaagcaatt cctagtccaa actctaatca tgttataacc | 1020 |
| ccaagctaac aaaggagcat ggtatccaaa aagcatggct gtaacaatag gtaaccctcc | 1080 |
| ccaaagtaat attgtaaaac aatgcaatat ttgaaaggaa acaatagagc atttgcaata | 1140 |
| taggatcaac atgttcaagt gacaagcatg acttgccttg ctctgctgct ggaggaacct | 1200 |
| cggcgactat ctcgaagtac actggagcgt cggaggaatc ggaatctagc gacatacaaa | 1260 |
| gcaaacattg caaaaaggct attagactac tgaaacagag gacaaaacca tttttaatgg | 1320 |
| attctacaca tttttcttga tttactgaga cttgaatggg cttaaacgga gctcggatga | 1380 |
| attagttatg aattttagaa gattcactat gtttgttact ataaaagaaa aaccttaaac | 1440 |
| aatttattgc gcaatatctt ccagggctga cgtcagcaca gagggggggt gcggcgccga | 1500 |
| catgcgggcc ccaccggtca gcggctcagg gggagcggtt caccgtggac cgggaccacg | 1560 |
| cgggtggtcc accgccggtc cacgggaccg acggcccaga tcgccctcgg gccgatcgga | 1620 |
| tgggcggcgg tagcggcagc cgccgggctc acctctgcat caaagccaac cggccgaccc | 1680 |
| gtgctaacgg cggcggcacg cgcgcgtcgc gttgagggcg acggcatccg gcggcggagg | 1740 |
| cgacggcaca aaggcgacgc gaaagcggcg gcagtcgaag gcgacggcgc gcgactcgaa | 1800 |
| gcggcggcgc ggcactaaag aggagaggaa ggggaaaaga ggggaggggc tgcctcaccg | 1860 |
| agggtggccg gcgcggagga agacgatggc gaacggcgac agcaagccgc gggaggatga | 1920 |
| cgtcggcggg cggacgagct tcggggtgcc ctagggaagg agaagagagg gattagagag | 1980 |
| aggggaatga accacggcga ccggaaacca tggccgaagg cggcggtaga gatggtgctc | 2040 |
| acccgtgcgc gtggggatgg gggctccggc gacgaacctc gacggaggag gggtggacga | 2100 |
| ggtggatctc ggccacgcga atcgaacggc ggcgatggcg caagacggcg gcgagcctag | 2160 |
| cggcggctag cggcggccgg agtagcggaa acggcggcgg caagaggttg cacagcgcgg | 2220 |
| gggcgatggg aggctcgaga gagtgcggcg aaatggggaa aaagtgagag gggacgacgg | 2280 |
| cgatgcttaa atagggggag aggggaccgg acgtggccgg gagaggcggg aatcaccggc | 2340 |
| cgacgcgggg gagtggggga ggagagagag gcgggattcg aaaatcgaat cccggccatc | 2400 |
| tcgggcgcgg gctcgagcgg gagagagagg ggagtgggcg cggaagacgg cgcacgcgcg | 2460 |
| ggcgtggacg acgtggcccg acaggtgggt gcggcggcgg tttcgggcgc ggcggcgcga | 2520 |
| gcgagggcgg cggtaggtgg gggaaggacc caacaggtgg gccccacctg tcggcgaccc | 2580 |
| cggagaggag ggcggcgggc ggcctggctg ggcctcggcc tgcggccggc ccagcaagga | 2640 |
| ggaggggaag gaatgggccg gcggcccatt cgaaaaaaag aggaaaaagg gagggaaaag | 2700 |
| aaaaagaaaa aaaagaaaaa ggaaaaaggg ttttccctgg aattaaaata ttgcttgctc | 2760 |
| cattttaatt ggttaaaatt atttctagag ctctgaaaat tccactaaaa atcctgttag | 2820 |
| tgaatttcga catgtagaac tcaaaaaaaa ttccacatgt cagatccgat tattatttgc | 2880 |
| attactttct tagggttttc tcctgatttg caccggtatt ttcttagggt catttataaa | 2940 |
| ttacaatttt ggcttgggag gaaaacttcg gggtgtgaca | 2980 |
| SEQ ID No. 2: | |
| cgccgaattg taggagttga caacgtcgtc gatgaagaag aatacaacca gaatcttcat | 60 |
| gtaagacctc acattgacct tgacgatgat ctccaagagc ctgtggcata tgctcattta | 120 |
| gatcatatgg aaggcataac agctaagcct taactaaacc gcagcgggat caccactcca | 180 |
| caggcatcct cgacggcacg gacgaagcct actcctcaga gaaaccttca tctgacctga | 240 |
| actcgaactc tggggttggg aaaagtagag caagactgag tactacccac tgtactcagc | 300 |
| aagtcatacc ggaatagagg tatgatgcag ggaatcatca aaaaaaaaac taaagtggtt | 360 |
| catttgcata aagcgagcat ttataaacag aagttgaaag gtttaaacag ttgtaataat | 420 |
| taatgattat taatcatccg ctgtccaacg ctaacctacg ttgcgacagg cccaaccatc | 480 |
| cacctaaacc atccaaatta aaagattaca ctaaggtgag actaatcacg gtgaatctgg | 540 |
| ttgaccgccc ataaccgcgg gcacggctat tcgaatagtt ttactctgat cagaggtgta | 600 |
| caactgtacc cacaagacac agccccacga cacgtttccg tgcgccgaca tgtcaccacg | 660 |
| acatatcgga aagaggccgt gacaggacca ttcgcataac cccctctaac caagcacacc | 720 |
| acacctcagg tttcaccccc actcctcgca aggtagcggg cagtcccctc tcgtgcctag | 780 |
| gtgaatccga aagccgcata ggccgtcgca gggcccatcc aaactccatc acgcccaccc | 840 |
| ttgcctggat gcgtctgcta ggggaaagct acactacaag cccagccgtt gcccatgctg | 900 |
| gcttgtggta agtacgttaa gttcttccag ggcatcccgc gaaccggtcc ttaactgcca | 960 |
| tgggtgcgac cagcaaaacc atgcacccac agcccaccat gtgattcatt ttatttaacc | 1020 |
| aacaccagag cggtggtact aatccaacat taccagaaga accaacagtc taaacattaa | 1080 |
| tatgattttc cccattgtga actagttgaa ctaagcatgg ctaagcaatt cctagtccaa | 1140 |
| actctaatca tgttataacc ccaagctaac aaaggagcat ggtatccaaa aagcatggct | 1200 |
| gtaacaatag gtaaccctcc ccaaagtaat attgtaaaac aatgcaatat ttgaaaggaa | 1260 |
| acaatagagc atttgcaata taggatcaac atgttcaagt gacaagcatg acttgccttg | 1320 |
| ctctgctgct ggaggaacct cggcgactat ctcgaagtac actggagcgt cggaggaatc | 1380 |
| ggaatctagc gacatacaaa gcaaacattg caaaaaggct attagactac tgaaacagag | 1440 |
| gacaaaacca tttttaatgg attctacaca tttttcttga tttactgaga cttgaatggg | 1500 |
| cttaaacgga gctcggatga attagttatg aattttagaa gattcactat gtttgttact | 1560 |
| ataaaagaaa aaccttaaac aatttattgc gcaatatctt ccagggctga cgtcagcaca | 1620 |
| gagggggggt gcggcgccga catgcgggcc ccaccggtca gcggctcagg gggagcggtt | 1680 |
| caccgtggac cgggaccacg cgggtggtcc accgccggtc cacgggaccg acggcccaga | 1740 |
| tcgccctcgg gccgatcgga tgggcggcgg tagcggcagc cgccgggctc acctctgcat | 1800 |
| caaagccaac cggccgaccc gtgctaacgg cggcggcacg cgcgcgtcgc gttgagggcg | 1860 |
| acggcatccg gcggcggagg cgacggcaca aaggcgacgc gaaagcggcg gcagtcgaag | 1920 |
| gcgacggcgc gcgactcgaa gcggcggcgc ggcactaaag aggagaggaa ggggaaaaga | 1980 |
| ggggaggggc tgcctcaccg agggtggccg gcgcggagga agacgatggc gaacggcgac | 2040 |
| agcaagccgc gggaggatga cgtcggcggg cggacgagct tcggggtgcc ctagggaagg | 2100 |
| agaagagagg gattagagag aggggaatga accacggcga ccggaaacca tggccgaagg | 2160 |
| cggcggtaga gatggtgctc acccgtgcgc gtggggatgg gggctccggc gacgaacctc | 2220 |
| gacggaggag gggtggacga ggtggatctc ggccacgcga atcgaacggc ggcgatggcg | 2280 |
| caagacggcg gcgagcctag cggcggctag cggcggccgg agtagcggaa acggcggcgg | 2340 |
| caagaggttg cacagcgcgg gggcgatggg aggctcgaga gagtgcggcg aaatggggaa | 2400 |
| aaagtgagag gggacgacgg cgatgcttaa atagggggag aggggaccgg acgtggccgg | 2460 |
| gagaggcggg aatcaccggc cgacgcgggg gagtggggga ggagagagag gcgggattcg | 2520 |
| aaaatcgaat cccggccatc tcgggcgcgg gctcgagcgg gagagagagg ggagtgggcg | 2580 |
| cggaagacgg cgcacgcgcg ggcgtggacg acgtggcccg acaggtgggt gcggcggcgg | 2640 |
| tttcgggcgc ggcggcgcga gcgagggcgg cggtaggtgg gggaaggacc caacaggtgg | 2700 |
| gccccacctg tcggcgaccc cggagaggag ggcggcgggc ggcctggctg ggcctcggcc | 2760 |
| tgcggccggc ccagcaagga ggaggggaag gaatgggccg gcggcccatt cgaaaaaaag | 2820 |
| aggaaaaagg gagggaaaag aaaaagaaaa aaaagaaaaa ggaaaaaggg ttttccctgg | 2880 |
| aattaaaata ttgcttgctc cattttaatt ggttaaaatt atttctagag ctctgaaaat | 2940 |
| tccactaaaa atcctgttag tgaatttcga catgtagaac tcaaaaaaaa ttccacatgt | 3000 |
| cagatccgat tattatttgc attactttct tagggttttc tcctgatttg caccggtatt | 3060 |
| ttcttagggt catttataaa ttacaatttt ggcttgggag gaaaacttcg gggtgtgaca | 3120 |
| tctactatag aactaacata tccattcact agttgtggca tggatataag tgaagcatcg | 3180 |
| cacaactctt ctttatcacg agaaacatca agcaaattgt tttgtgacaa aagtatttca | 3240 |
| gcaattgatt ccactatggg ttgctccaac atagcataag ggtgtgttcg caggtggagg | 3300 |
| atgggaaccc atccctcctg cacgcaaatg gagcagcctt ttaacacgta attaattaag | 3360 |
| tattagttaa ttttttttga aaaaatggat caatatgaat ttttaaagca actttcgtat | 3420 |
| ataaaatttt ttgaaaaaga tgcaccgttt agtagtttga aaagcgtgca tgcggaaaac | 3480 |
| gagagaggtg ggttgggaag agggagtgcc aaactcagcc taagtagtgg acatagttgg | 3540 |
| cacatcataa atatgcatac cttgagtagt tgtagcacca ttatcattac cttttttctc | 3600 |
| atcttcaga | 3609 |
| SEQ ID No. 3: | |
| acttgacaat cgatccaact agc | 23 |
| SEQ ID No. 4: | |
| cgaagctttg ctgatcggg | 19 |
| SEQ ID No. 5: | |
| cgccgaattg taggagttg | 19 |
| SEQ ID No. 6: | |
| ggatggttta ggtggatgg | 19 |

The present invention has been described in detail above. For those skilled in the art, without departing from the spirit and scope of the present invention, and without unnecessary experiments, the present invention can be practiced in a wider range under equivalent parameters, concentrations and conditions. While specific embodiments of the present invention have been shown, it should be understood that the present invention can be further modified. In a word, according to the principles of the present invention, this present application intends to include any changes, uses or improvements to the present invention, including changes made by using conventional techniques known in the art and departing from the disclosed scope of this application.

Uses of some of the essential features can be applied within the scope of the appended claims below.

### INDUSTRIAL APPLICATION

The present invention found that the DNA fragment in the 8899129-9307609 region of rice chromosome 7 is related to the cadmium accumulation of rice grains. The DNA fragment sequence here is homozygous rice with SEQ ID No.1 in the sequence listing having low grain cadmium accumulation traits when grown in highly cadmium-polluted fields. Therefore, the DNA fragment can be introduced into other backgrounds by hybridization, backcross and other breeding methods to breed new rice varieties with low grain cadmium accumulation.

1-15. (canceled)

16. A method for identifying or assisting in the identification of low cadmium accumulation traits in rice grains, comprising the use of a substance for detecting a molecular marker of cadmium accumulation in rice, wherein the molecular marker of cadmium accumulation in rice is a DNA fragment in a 8899129-9307609 region of a rice chromosome 7; the molecular marker of cadmium accumulation in rice is lcrf1 or lcrf2, and the lcrf1 is M1) or M2):
M1) a DNA fragment shown in the 8899129-9307609 of the rice chromosome 7, M2) ;,',,a DNA fragment that undergoes substitution and/or deletion and/or addition of one or several nucleotides or DNA fragments to M1) and has 75% or more identity to M1);
the lcrf2 is N1) or N2):
N1) a DNA fragment shown in SEQ ID No.1 in the sequence listing, N2) a DNA fragment that undergoes substitution and/or deletion and/or addition of one or several nucleotides or DNA fragments to N1) and has 75% or more identity to N1).

17. The method according to claim 16, characterized in that the substance for detecting a molecular marker of cadmium accumulation in rice is a PCR primer capable of distinguishing the molecular marker of cadmium accumulation in rice as the lcrf1 and the lcrf2.

18. The method according to claim 17, characterized in that the PCR primer is part or all of the following (a1), (a2), (a3) and (a4):
(a1) is (b1) or (b2) or (b3) as follows:
(b1) a single-stranded DNA molecule shown in SEQ ID No.3 of the sequence listing;
(b2) a single-stranded DNA molecule that undergoes substitution and/or deletion and/or addition of one or several nucleotides to SEQ ID No. 3 and has 75% or more identity to (b1);
(b3) a DNA molecule that hybridizes under stringent conditions to a nucleotide sequence defined in (b1) or (b2);
(a2) is (c1) or (c2) or (c3) as follows:
(c1) a single-stranded DNA molecule shown in SEQ ID No.4 of the sequence listing;
(c2) a single-stranded DNA molecule that undergoes substitution and/or deletion and/or addition of one or several nucleotides to SEQ ID No. 4 and has 75% or more identity to (c1);
(c3) a DNA molecule that hybridizes under stringent conditions to a nucleotide sequence defined in (c1) or (c2);
(a3) is (d1) or (d2) or (d3) as follows:
(d1) a single-stranded DNA molecule shown in SEQ ID No.5 of the sequence listing;
(d2) a single-stranded DNA molecule that undergoes substitution and/or deletion and/or addition of one or several nucleotides to SEQ ID SEQ ID No.5 and has 75% or more identity to (d1);
(d3) a DNA molecule that hybridizes under stringent conditions to a nucleotide sequence defined in (d1) or (d2);
(a4) is (e1) or (e2) or (e3) as follows:
(e1) a single-stranded DNA molecule shown in SEQ ID No. 6 of the sequence listing;
(e2) a single-stranded DNA molecule that undergoes substitution and/or deletion and/or addition of one or several nucleotides to SEQ ID No. 6 and has 75% or more identity to (e1);
(e3) a DNA molecule that hybridizes under stringent conditions to a nucleotide sequence defined in (e1) or (e2).

19. The method according to claim 16, characterized in that the method comprises:
detecting the DNA fragment in a 8899129-9307609 region of a chromosome 7 of rice to be tested, and determining the low grain cadmium accumulation traits of the rice to be tested according to the DNA fragment in the 8899129-9307609 region of the chromosome 7 of the rice to be tested; the DNA fragment in the 8899129-9307609 region of rice chromosome 7 is the lcrf1 or the lcrf2; the cadmium content in grains of the homozygous rice containing the lcrf2 is lower or candidate lower than that of heterozyous rice containing the lcrf1 and the lcrf2, and the cadmium content in grains of homozygous rice containing the lcrf2 is lower or candidate lower than that of homozygous rice containing lcrf1.

20. Any of the following methods:
(1)a method in the preparation of a product for identifying or assisting in the identification of low cadmium accumulation traits in rice grains, comprising the use of the substance for detecting a molecular marker of cadmium accumulation in rice according to claim 16;
(2) a method in rice breeding or preparing a rice breeding product, comprising the use of the substance for detecting a molecular marker of cadmium accumulation in rice according to claim 16;
(3) a method in rice breeding, comprising the method according to claim 16;
(4) a rice breeding method, comprising: detecting a DNA fragment in a 8899129-9307609 region of a chromosome 7 in a rice genome, and selecting a DNA fragment in the 8899129-9307609 region of the chromosome 7 as a homozygous or heterozygous rice for lcrf2 according to claim 16 as a parent for breeding.

21. The method according to claim 20, characterized in that the substance for detecting a molecular marker of cadmium accumulation in rice is a PCR primer capable of distinguishing the molecular marker of cadmium accumulation in rice as the lcrf1 and the lcrf2.

22. The method according to claim 21, characterized in that the PCR primer is part or all of the following (a1), (a2), (a3) and (a4):
(a1) is (b1) or (b2) or (b3) as follows:
(b1) a single-stranded DNA molecule shown in SEQ ID No.3 of the sequence listing;
(b2) a single-stranded DNA molecule that undergoes substitution and/or deletion and/or addition of one or several nucleotides to SEQ ID No. 3 and has 75% or more identity to (b1);
(b3) a DNA molecule that hybridizes under stringent conditions to a nucleotide sequence defined in (b1) or (b2);
(a2) is (c1) or (c2) or (c3) as follows:
(c1) a single-stranded DNA molecule shown in SEQ ID No.4 of the sequence listing;
(c2) a single-stranded DNA molecule that undergoes substitution and/or deletion and/or addition of one or several nucleotides to SEQ ID No. 4 and has 75% or more identity to (c1);
(c3) a DNA molecule that hybridizes under stringent conditions to a nucleotide sequence defined in (c1) or (c2);
(a3) is (d1) or (d2) or (d3) as follows:
(d1) a single-stranded DNA molecule shown in SEQ ID No.5 of the sequence listing;
(d2) a single-stranded DNA molecule that undergoes substitution and/or deletion and/or addition of one or several nucleotides to SEQ ID SEQ ID No.5 and has 75% or more identity to (d1);
(d3) a DNA molecule that hybridizes under stringent conditions to a nucleotide sequence defined in (d1) or (d2);
(a4) is (e1) or (e2) or (e3) as follows:
(e1) a single-stranded DNA molecule shown in SEQ ID No. 6 of the sequence listing;
(e2) a single-stranded DNA molecule that undergoes substitution and/or deletion and/or addition of one or several nucleotides to SEQ ID No. 6 and has 75% or more identity to (e1);
(e3) a DNA molecule that hybridizes under stringent conditions to a nucleotide sequence defined in (e1) or (e2).

23. The method according to claim 20, characterized in that the rice breeding is to cultivate rice with low grain cadmium accumulation.

24. Any of the following substances:
(1) the substance for detecting a molecular marker of cadmium accumulation in rice according to any one according to claim 16;
(2) the lcrf2 according to claim 16.


SEQUENCE LISTING (6 sequences)

SEQ ID NO: 1 - DNA, 2980 bp, Oryza sativa
agctaagccttaactaaaccgcagcgggatcaccactccacaggcatcctcgacggcacggacgaagcctactcctcagagaaaccttcatctgacctgaactcgaactctggggttgggaaaagtagagcaagactgagtactacccactgtactcagcaagtcataccggaatagaggtatgatgcagggaatcatcaaaaaaaaaactaaagtggttcatttgcataaagcgagcatttataaacagaagttgaaaggtttaaacagttgtaataattaatgattattaatcatccgctgtccaacgctaacctacgttgcgacaggcccaaccatccacctaaaccatccaaattaaaagattacactaaggtgagactaatcacggtgaatctggttgaccgcccataaccgcgggcacggctattcgaatagttttactctgatcagaggtgtacaactgtacccacaagacacagccccacgacacgtttccgtgcgccgacatgtcaccacgacatatcggaaagaggccgtgacaggaccattcgcataaccccctctaaccaagcacaccacacctcaggtttcacccccactcctcgcaaggtagcgggcagtcccctctcgtgcctaggtgaatccgaaagccgcataggccgtcgcagggcccatccaaactccatcacgcccacccttgcctggatgcgtctgctaggggaaagctacactacaagcccagccgttgcccatgctggcttgtggtaagtacgttaagttcttccagggcatcccgcgaaccggtccttaactgccatgggtgcgaccagcaaaaccatgcacccacagcccaccatgtgattcattttatttaaccaacaccagagcggtggtactaatccaacattaccagaagaaccaacagtctaaacattaatatgattttccccattgtgaactagttgaactaagcatggctaagcaattcctagtccaaactctaatcatgttataaccccaagctaacaaaggagcatggtatccaaaaagcatggctgtaacaataggtaaccctccccaaagtaatattgtaaaacaatgcaatatttgaaaggaaacaatagagcatttgcaatataggatcaacatgttcaagtgacaagcatgacttgccttgctctgctgctggaggaacctcggcgactatctcgaagtacactggagcgtcggaggaatcggaatctagcgacatacaaagcaaacattgcaaaaaggctattagactactgaaacagaggacaaaaccatttttaatggattctacacatttttcttgatttactgagacttgaatgggcttaaacggagctcggatgaattagttatgaattttagaagattcactatgtttgttactataaaagaaaaaccttaaacaatttattgcgcaatatcttccagggctgacgtcagcacagagggggggtgcggcgccgacatgcgggccccaccggtcagcggctcagggggagcggttcaccgtggaccgggaccacgcgggtggtccaccgccggtccacgggaccgacggcccagatcgccctcgggccgatcggatgggcggcggtagcggcagccgccgggctcacctctgcatcaaagccaaccggccgacccgtgctaacggcggcggcacgcgcgcgtcgcgttgagggcgacggcatccggcggcggaggcgacggcacaaaggcgacgcgaaagcggcggcagtcgaaggcgacggcgcgcgactcgaagcggcggcgcggcactaaagaggagaggaaggggaaaagaggggaggggctgcctcaccgagggtggccggcgcggaggaagacgatggcgaacggcgacagcaagccgcgggaggatgacgtcggcgggcggacgagcttcggggtgccctagggaaggagaagagagggattagagagaggggaatgaaccacggcgaccggaaaccatggccgaaggcggcggtagagatggtgctcacccgtgcgcgtggggatgggggctccggcgacgaacctcgacggaggaggggtggacgaggtggatctcggccacgcgaatcgaacggcggcgatggcgcaagacggcggcgagcctagcggcggctagcggcggccggagtagcggaaacggcggcggcaagaggttgcacagcgcgggggcgatgggaggctcgagagagtgcggcgaaatggggaaaaagtgagaggggacgacggcgatgcttaaatagggggagaggggaccggacgtggccgggagaggcgggaatcaccggccgacgcgggggagtgggggaggagagagaggcgggattcgaaaatcgaatcccggccatctcgggcgcgggctcgagcgggagagagaggggagtgggcgcggaagacggcgcacgcgcgggcgtggacgacgtggcccgacaggtgggtgcggcggcggtttcgggcgcggcggcgcgagcgagggcggcggtaggtgggggaaggacccaacaggtgggccccacctgtcggcgaccccggagaggagggcggcgggcggcctggctgggcctcggcctgcggccggcccagcaaggaggaggggaaggaatgggccggcggcccattcgaaaaaaagaggaaaaagggagggaaaagaaaaagaaaaaaaagaaaaaggaaaaagggttttccctggaattaaaatattgcttgctccattttaattggttaaaattatttctagagctctgaaaattccactaaaaatcctgttagtgaatttcgacatgtagaactcaaaaaaaattccacatgtcagatccgattattatttgcattactttcttagggttttctcctgatttgcaccggtattttcttagggtcatttataaattacaattttggcttgggaggaaaacttcggggtgtgaca

SEQ ID NO: 2 - DNA, 3609 bp, Oryza sativa
cgccgaattgtaggagttgacaacgtcgtcgatgaagaagaatacaaccagaatcttcatgtaagacctcacattgaccttgacgatgatctccaagagcctgtggcatatgctcatttagatcatatggaaggcataacagctaagccttaactaaaccgcagcgggatcaccactccacaggcatcctcgacggcacggacgaagcctactcctcagagaaaccttcatctgacctgaactcgaactctggggttgggaaaagtagagcaagactgagtactacccactgtactcagcaagtcataccggaatagaggtatgatgcagggaatcatcaaaaaaaaaactaaagtggttcatttgcataaagcgagcatttataaacagaagttgaaaggtttaaacagttgtaataattaatgattattaatcatccgctgtccaacgctaacctacgttgcgacaggcccaaccatccacctaaaccatccaaattaaaagattacactaaggtgagactaatcacggtgaatctggttgaccgcccataaccgcgggcacggctattcgaatagttttactctgatcagaggtgtacaactgtacccacaagacacagccccacgacacgtttccgtgcgccgacatgtcaccacgacatatcggaaagaggccgtgacaggaccattcgcataaccccctctaaccaagcacaccacacctcaggtttcacccccactcctcgcaaggtagcgggcagtcccctctcgtgcctaggtgaatccgaaagccgcataggccgtcgcagggcccatccaaactccatcacgcccacccttgcctggatgcgtctgctaggggaaagctacactacaagcccagccgttgcccatgctggcttgtggtaagtacgttaagttcttccagggcatcccgcgaaccggtccttaactgccatgggtgcgaccagcaaaaccatgcacccacagcccaccatgtgattcattttatttaaccaacaccagagcggtggtactaatccaacattaccagaagaaccaacagtctaaacattaatatgattttccccattgtgaactagttgaactaagcatggctaagcaattcctagtccaaactctaatcatgttataaccccaagctaacaaaggagcatggtatccaaaaagcatggctgtaacaataggtaaccctccccaaagtaatattgtaaaacaatgcaatatttgaaaggaaacaatagagcatttgcaatataggatcaacatgttcaagtgacaagcatgacttgccttgctctgctgctggaggaacctcggcgactatctcgaagtacactggagcgtcggaggaatcggaatctagcgacatacaaagcaaacattgcaaaaaggctattagactactgaaacagaggacaaaaccatttttaatggattctacacatttttcttgatttactgagacttgaatgggcttaaacggagctcggatgaattagttatgaattttagaagattcactatgtttgttactataaaagaaaaaccttaaacaatttattgcgcaatatcttccagggctgacgtcagcacagagggggggtgcggcgccgacatgcgggccccaccggtcagcggctcagggggagcggttcaccgtggaccgggaccacgcgggtggtccaccgccggtccacgggaccgacggcccagatcgccctcgggccgatcggatgggcggcggtagcggcagccgccgggctcacctctgcatcaaagccaaccggccgacccgtgctaacggcggcggcacgcgcgcgtcgcgttgagggcgacggcatccggcggcggaggcgacggcacaaaggcgacgcgaaagcggcggcagtcgaaggcgacggcgcgcgactcgaagcggcggcgcggcactaaagaggagaggaaggggaaaagaggggaggggctgcctcaccgagggtggccggcgcggaggaagacgatggcgaacggcgacagcaagccgcgggaggatgacgtcggcgggcggacgagcttcggggtgccctagggaaggagaagagagggattagagagaggggaatgaaccacggcgaccggaaaccatggccgaaggcggcggtagagatggtgctcacccgtgcgcgtggggatgggggctccggcgacgaacctcgacggaggaggggtggacgaggtggatctcggccacgcgaatcgaacggcggcgatggcgcaagacggcggcgagcctagcggcggctagcggcggccggagtagcggaaacggcggcggcaagaggttgcacagcgcgggggcgatgggaggctcgagagagtgcggcgaaatggggaaaaagtgagaggggacgacggcgatgcttaaatagggggagaggggaccggacgtggccgggagaggcgggaatcaccggccgacgcgggggagtgggggaggagagagaggcgggattcgaaaatcgaatcccggccatctcgggcgcgggctcgagcgggagagagaggggagtgggcgcggaagacggcgcacgcgcgggcgtggacgacgtggcccgacaggtgggtgcggcggcggtttcgggcgcggcggcgcgagcgagggcggcggtaggtgggggaaggacccaacaggtgggccccacctgtcggcgaccccggagaggagggcggcgggcggcctggctgggcctcggcctgcggccggcccagcaaggaggaggggaaggaatgggccggcggcccattcgaaaaaaagaggaaaaagggagggaaaagaaaaagaaaaaaaagaaaaaggaaaaagggttttccctggaattaaaatattgcttgctccattttaattggttaaaattatttctagagctctgaaaattccactaaaaatcctgttagtgaatttcgacatgtagaactcaaaaaaaattccacatgtcagatccgattattatttgcattactttcttagggttttctcctgatttgcaccggtattttcttagggtcatttataaattacaattttggcttgggaggaaaacttcggggtgtgacatctactatagaactaacatatccattcactagttgtggcatggatataagtgaagcatcgcacaactcttctttatcacgagaaacatcaagcaaattgttttgtgacaaaagtatttcagcaattgattccactatgggttgctccaacatagcataagggtgtgttcgcaggtggaggatgggaacccatccctcctgcacgcaaatggagcagccttttaacacgtaattaattaagtattagttaattttttttgaaaaaatggatcaatatgaatttttaaagcaactttcgtatataaaattttttgaaaaagatgcaccgtttagtagtttgaaaagcgtgcatgcggaaaacgagagaggtgggttgggaagagggagtgccaaactcagcctaagtagtggacatagttggcacatcataaatatgcataccttgagtagttgtagcaccattatcattaccttttttctcatcttcaga

SEQ ID NO: 3 - DNA, 23 bp, Artificial sequence
  Synthesized
acttgacaatcgatccaactagc

SEQ ID NO: 4 - DNA, 19 bp, Artificial sequence
  Synthesized
cgaagctttgctgatcggg

SEQ ID NO: 5 - DNA, 19 bp, Artificial sequence
  Synthesized
cgccgaattgtaggagttg

SEQ ID NO: 6 - DNA, 19 bp, Artificial sequence
  Synthesized
ggatggtttaggtggatgg